(19) United States
(12) Patent Application Publication
(10) Pub. No.: US 2018/0289503 A1
(43) Pub. Date: Oct. 11, 2018

(54) OBLIQUE INTERBODY DEVICE

(71) Applicant: Spineology Inc., St. Paul, MN (US)

(72) Inventors: Troy KNAPP, Glenwood City, WI (US); Garrett GANSKE, Lino Lakes, MN (US); Tejpaul PANNU, Birmingham, MI (US); Clifford TRIBUS, Madison, WI (US); Jeremy SHORE, Peabody, MA (US)

(21) Appl. No.: 15/942,407

(22) Filed: Mar. 30, 2018

Related U.S. Application Data
(60) Provisional application No. 62/480,177, filed on Mar. 31, 2017.

(51) Int. Cl. A61F 2/44; A61B 17/86

(52) U.S. Cl. CPC A61F 2/4455; A61F 2002/30331; A61B 17/8605; A61B 17/8625

(57) ABSTRACT

An oblique interbody spacer system includes a spacer body, a fixation plate and a plurality of angled fixation channels. The fixation plate is integrated with the spacer body such that the spacer body and fixation plate form a singular component for insertion. The implant can be affixed to the superior and inferior vertebral bodies with screws obliquely (in the range of about 30° from straight anterior).

### PRIORITY

This application claims the benefit of U.S. Provisional Application Ser. No. 62/480,177, filed on Mar. 31, 2017, which is hereby incorporated herein by reference in its entirety.

### FIELD

The present invention generally relates to an interbody vertebral spacer device designed to be inserted antero-laterally. More particularly, the present invention relates to an antero-lateral interbody device having integrated screw fixation.

### BACKGROUND

It is desirable to avoid the anterior vessels when inserting an interbody vertebral spacer, especially in levels above L5/S1, where the vessels can be immobile. A common insertion approach is to insert a device directly anteriorly. This approach requires the surgeon to sweep the vessels to the side. One can avoid moving the vessels by inserting an implant off to the side, or obliquely, and around the vessels. Oblique implants exist, but none include integrated screw fixation.

### SUMMARY

The disclosure includes an oblique interbody device having an integrated fixation faceplate that allows the implant to be affixed to the superior and inferior vertebral bodies with screws obliquely (in the range of about 30° from straight anterior).

The disclosure also includes an oblique interbody spacer system, including a spacer body, a fixation plate and a plurality of angled fixation channels. The fixation plate is integrated with the spacer body such that the spacer body and fixation plate form a singular component for insertion.

The plurality of angled fixation channels can include a first angled fixation channel that is angled differently with respect to the spacer body than the a second angled fixation channel. A plurality of fixation members, such as screws, can each be disposed into a respective one of the plurality of angled fixation channels. A fixation member backout preventer can be provided to the fixation plate. Each of the plurality of angled fixation channels can define a different trajectory.

The disclosure further includes a method of placing an implant into an intervertebral space of a patient. An oblique approach to the spine is employed to place the implant and an integrated fixation plate simultaneously into the intervertebral space. Fixation screws are inserted obliquely prior to rolling the patient over and installing a posterior hardware component.

The detailed technology and preferred embodiments implemented for the subject invention are described in the following paragraphs accompanying the appended drawings for people skilled in this field to well appreciate the features of the claimed invention. It is understood that the features mentioned hereinbefore and those to be commented on hereinafter may be used not only in the specified combinations, but also in other combinations or in isolation, without departing from the scope of the present invention.

### BRIEF DESCRIPTION OF THE DRAWINGS

FIG. 1 depicts a conventional approach to implant insertion.

FIG. 2 depicts an embodiment of the present invention according to an oblique approach to implant insertion.

FIG. 3 depicts a top view of an implant according to certain embodiments.

FIG. 4 depicts a perspective of an implant according to certain embodiments.

While the invention is amenable to various modifications and alternative forms, specifics thereof have been shown by way of example in the drawings and will be described in detail. It should be understood, however, that the intention is not to limit the invention to the particular example embodiments described. On the contrary, the invention is to cover all modifications, equivalents, and alternatives falling within the spirit and scope of the invention as defined by the appended claims. For illustrative purposes, cross-hatching, dashing or shading in the figures is provided to demonstrate sealed portions and/or integrated regions or devices for the package.

### DETAILED DESCRIPTION

In the following descriptions, the present invention will be explained with reference to example embodiments thereof. However, these embodiments are not intended to limit the present invention to any specific example, embodiment, environment, applications or particular implementations described in these embodiments. Therefore, description of these embodiments is only for purpose of illustration rather than to limit the present invention. It should be appreciated that, in the following embodiments and the attached drawings, elements unrelated to the present invention are omitted from depiction; and dimensional relationships among individual elements in the attached drawings are illustrated only for ease of understanding, but not to limit the actual scale.

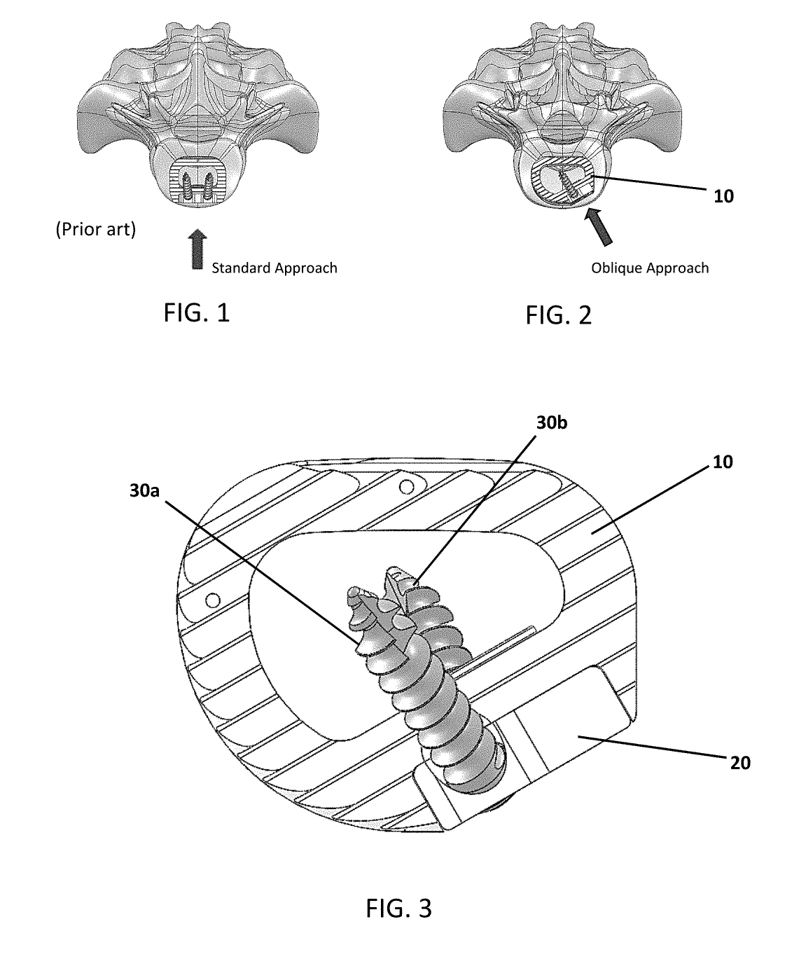

Referring to FIG. 1, a standard or conventional anterior approach to the spine is depicted.

FIG. 2 depicts an embodiment of the present invention wherein the implant 10 is introduced from an oblique approach to the spine.

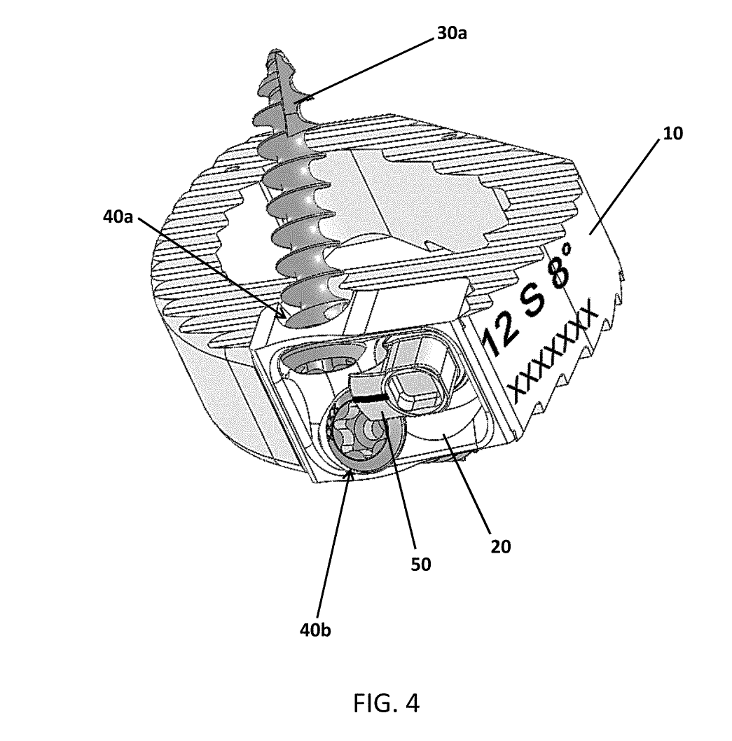

Referring now to FIGS. 3-4, a spacer body 10 that can be used with the oblique approach is shown. Body 10 includes an integrated fixation plate 20 thereon, making body 10 and plate 20 one singular component for insertion. Thus, the surgeon need not separately dispose a plate on the implant body post-insertion.

FIG. 3 further depicts fixation members 30a and 30b. According to this embodiment, fixation members may be screws. However, other suitable types of fixation members may also be used.

As can be seen in FIG. 4, the face plate 20 includes angled fixation channels 40a and 40b. Angled channels 40a and 40b guide the trajectory of fixation members 30a and 30b into the surgeon's desired position.

A backout preventer 50 is also provided to the plate 20. This prevents the screws 30a and 30b from unintentionally backing out of the patient's anatomy.

In use, because plate 20 is an integral part of implant 10, the surgeon places implant 10 and fixation plate 20 simultaneously, in one step. In contrast, conventional oblique implants require the fixation plate to be placed separately from the implant as a multi-step procedure. The conventional process may lead to patient tissue being trapped between the plate and the implant and also requires a second insertion step prior to screw placement. The present invention is configured to address these drawbacks of the conventional procedure.

Once implant 10 with integrated fixation plate 20 is inserted, the surgeon may place fixation screws 30a and 30b through the angled fixation channels 40a and 40b. Screws 30a and 30b allow for better purchase than bladed systems into the patient's cortical bone, and screws allow for easier revision than bladed systems.

The present invention may be embodied in other specific forms without departing from the spirit or essential attributes thereof, and it is, therefore, desired that the present embodiment be considered in all respects as illustrative and not restrictive. Those skilled in the art may recognize other equivalents to the specific embodiment described herein which equivalents are intended to be encompassed by the claims attached hereto.

What is claimed is:

1. An oblique interbody spacer system with integrated fixation, comprising:
a spacer body;
a fixation plate integrated with the spacer body such that the spacer body and fixation plate form a singular component for insertion; and
a plurality of angled fixation channels defined within the spacer body and fixation plate.

2. The system of claim 1, wherein the plurality of angled fixation channels include a first angled fixation channel that is angled differently with respect to the spacer body than the a second angled fixation channel.

3. The system of claim 2, further comprising a plurality of fixation members corresponding to the plurality of angled fixation channels.

4. The system of claim 3, wherein each of the plurality of fixation members is a screw.

5. The system of claim 1, further comprising a fixation member backout preventer provided to the fixation plate.

6. The system of claim 1, wherein each of the plurality of angled fixation channels defines a different trajectory.

7. A method of placing an implant into an intervertebral space of a patient, the method comprising:
employing an oblique approach to the spine to place the implant and an integrated fixation plate simultaneously into the intervertebral space; and
inserting fixation screws obliquely prior to rolling the patient over and installing a posterior hardware component.
